(19) United States
(12) Patent Application Publication
(10) Pub. No.: US 2017/0071791 A1
(43) Pub. Date: Mar. 16, 2017

(54) TOROIDAL GLAUCOMA DRAINAGE DEVICE

(71) Applicant: TEL HASHOMER MEDICAL RESEARCH INFRASTRUCTURE AND SERVICES LTD., Ramat Gan (IL)

(72) Inventor: Ilia PIVEN, Petach Tikwa (IL)

(21) Appl. No.: 15/122,946

(22) PCT Filed: Mar. 5, 2015
(86) PCT No.: PCT/IL2015/050237

§ 371 Date: Sep. 1, 2016

(30) Foreign Application Priority Data
Mar. 10, 2014 (IL) 231435

(51) Int. Cl. A61F 9/007; A61F 9/008

(52) U.S. Cl. CPC A61F 9/00781; A61F 9/008

(57) ABSTRACT

The invention provides an intraocular drainage implant whose drainage capacity is regulated by opening a plurality of laser-sensitive spots. The implant is useful for fine regulation of the eye fluid outflow in handling glaucoma.

### FIELD OF THE INVENTION

The present invention relates to an implantable device for ocular drainage useful in handling glaucoma. The device and methods of use thereof enable fine regulation of the eye fluid outflow.

### BACKGROUND OF THE INVENTION

Glaucoma is a widespread eye disease, afflicting more than two million people in the U.S. alone and causing blindness in 120,000 of them. The only modifiable risk factor of glaucoma is high intraocular pressure (IOP), thus all the treatment options try to control it. Eye drops or oral medications are employed to lower the eye pressure, but they often do not succeed in controlling eye pressure or they result in hardly tolerable side effects, necessitating surgery. The surgical procedures usually include trabeculectomy aiming at opening the full thickness of the drainage area, or laser trabeculoplasty that partially opens the drainage area. Trabeculectomy, with small modifications, has been used for hundred of years. Many of the serious side effects or complications that occur with the procedure involve blister formation, blebs, which cause leakage and may eventually cause blindness. Some drugs used to prevent post-operational scarring unfortunately cause late-onset leaking blebs, requiring lifelong monitoring after surgery. In certain patient groups, such as young patients, patients who have taken multiple drugs or have had an inflammatory disease, or who have had cataract surgery, scars form around the incision and close up the drainage channels; the pressure increases again and special procedures must be applied to restore the drainage. An alternative method to trabeculectomy, laser trabeculoplasty, involves burning tiny holes into the tissue by laser, whereby creating tiny scars which increase fluid outflow, but the patients might still need their medications every day, and many of them need additional surgery or re-medications within several years. In about one third of the patients after the above surgery procedures, pressure temporarily increases, and in some cases the increase is permanent with the vision loss. Another complication in up to one third of patients is developing adhesive-like substances which may glue the iris to the cornea. When glaucoma does not respond to standard procedures, the drainage implants, also called tube shunts, are used. Said implants are also needed in cases when glaucoma is caused by swelling in the iris or by abnormal vessel formations, during iridocorneal endothelial (ICE) syndrome, etc. In general, the procedure usually involves an implant of about a half inch silicone tube, inserted into the eye's anterior chamber and draining the fluid out. Complications after such implantation may include cataract, detached retina, breakdown of the cornea, bleeding, and hypotony, the latter occurring when the outflow is excessive. Other risks and problems include eye movement disorders, such as double vision and strabismus, or implant blockage, requiring additional surgery. Some techniques are too risky and are reserved only for people with end-stage glaucoma. Many techniques require extraordinary surgical skill, and even if performed perfectly, they do not always sufficiently handle the IOPs. Special problems, like for example combined cataract with glaucoma, require special attitudes, and despite their variability, the available techniques frequently cannot provide an efficient solution.

Known glaucoma drainage devices have usually a single tube that, if successfully implanted, has a free end in the anterior chamber and another end outside the eye. The free inner end has a great potential to multiple complications—it can move, be displaced and eventually touch surfaces of the cornea and iris resulting in many problems, like corneal endothelial cells loss, pigment dispersion syndrome, inflammation, hemorrhages, failure, cataracts, etc.; the outer parts, beside apparent inconvenience, are exposed to potential infections and other perils. Undesired phenomena include, on one hand, blocking of the drainage flow, and, on the other hand, excessive drainage; the liquid outflow can be adjusted to the observed clinical IOP only with great difficulties. U.S. Pat. No. 3,788,327 describes a device in the form of a tube to be implanted into the anterior eye chamber through the cornea and releasing fluid through a valve means onto the eye surface. U.S. Pat. No. 4,936,825 describes a method for reducing the pressure in the eye via implanting a porous cord arranged in a loop extending through the cornea onto the eye surface. Both devices release the fluid onto the eye surface situated outside the patient's body. Among other drawbacks, said available devices are disadvantageously exposed on the cornea surface, and moreover have very limited and mostly unpredictable means to control the eye fluid flows.

It is therefore an object of this invention to provide a glaucoma drainage device without the drawbacks of the known devices.

It is another object of this invention to provide a glaucoma drainage device (GDD) with enables a sufficient flow of the liquid out of the eye, whereby lowering the intraocular pressure.

It is still another object of this invention to provide a GDD of which flow permeability can be tuned in accordance with the postoperatively observed IOP.

It is a further object of this invention to provide a GDD which enables to increase the flow rate of the liquid from the eye by a simpler an safer procedure.

It is a still further object of this invention to provide a GDD which does not exhibit free tube ends.

It is an aim of this invention to provide a GDD of which no parts are exposed on the outer surface of the cornea.

It is another aim of this invention to provide a GDD which has a fixed position toward the eye parts and which does not reach, touch or irritate the cornea, the iris or lens.

This invention also aims at providing a method for handling the intraocular pressure by implanting a drainage device which cannot move toward the eye parts, which is not exposed on the visible eye surface, and which has a drainage capacity adjustable in accordance with the observed IOP.

Other objects and advantages of present invention will appear as description proceeds.

### SUMMARY OF THE INVENTION

The invention provides an intraocular drainage implant with adjustable drainage capacity, comprising an outer surface impermeable to aqueous fluids with a plurality of laser-sensitive spots permeable to aqueous fluids after laser irradiation. Said implant is preferably a glaucoma drainage device. In a preferred embodiment, the device comprises a tubing whose ends are attached to each other by means of a lock, to form a closed loop. In a preferred embodiment of the invention, the implanted device has essentially a toroidal shape. The device is preferably implanted in the anterior chamber near or through the limbus of an eye. Said implant usually comprises a tubing closed in a loop having a narrowed site, a neck, to be used for fixation suture. The implant of the invention has a fixed position within the eye, preferably near the limbus of an eye, wherein a part of said loop is located within the anterior chamber of said eye, and wherein said neck is sutured to the sclera of said eye. In one embodiment, said lock comprises a T-tube connected to a linear tube for shunting eye fluid from said loop to an extraocularly situated plate. The term T-tube is intended to cover any branching means, for example a Y-shaped tube or a T-shaped tube, or other means. In one embodiment, the implant of the invention comprises a tubing closed in a loop by a T-shaped lock, a linear tube connected to the loop via said lock, and an extraocularly situated plate (pad) connected with said linear tube, wherein a part of said loop is located within the anterior chamber of said eye, and wherein said neck and said plate are sutured to the sclera, the plate being onto the external surface of the sclera. The implant of the invention drains the eye fluid out of the anterior chamber, wherein a part of said loop is located inside the anterior chamber near the limbus of said eye and a part of said loop is located outside said anterior chamber. The invention enables to adjust the drainage capacity of the implant; the loop implant preferably has an outer surface impermeable to aqueous fluids with a plurality of laser-sensitive spots permeable to aqueous fluids after laser irradiation, wherein said eye fluid enters to said loop part which is located in the anterior chamber, via openings created from at least a part of said plurality of spots, and wherein said fluid is shunted via said loop out of the anterior chamber. The implant may be sequentially irradiated by laser in several steps, so that a greater part of said plurality of spots is opened to aqueous fluids after each step, thereby optimizing the volume of fluid to be continually removed from the eye. When using the term aqueous fluid, biological fluids are included, such as aqueous humor.

The invention is directed to a glaucoma drainage device for intraocular implanting, comprising a tubing having an outer surface impermeable to aqueous fluids with a plurality of laser-sensitive spots permeable to aqueous fluids after laser irradiation, wherein free ends of the tubing are attached to each other by means of a lock to form a closed loop, the device being implanted in the anterior chamber near the limbus of an eye and being sutured to the sclera to ensure its fixed position within the eye, said lock optionally comprising a T-tube to connect said loop with an extraocularly situated plate via an essentially linear connector, wherein a part of said loop is located within the anterior chamber of said eye shunting fluid out of the anterior chamber, the volume of said fluid being affected by the fraction of said spots being converted to openings by said irradiation, the fraction being optimized by sequentially irradiating the device in several increments. The glaucoma drainage device according to the invention is mainly used in managing refractory glaucoma.

The invention provides a method of treating refractory glaucoma in an eye in need of such treatment, comprising i) providing an intraocular tubular implant having an outer surface impermeable to aqueous fluids with a plurality of laser-sensitive spots permeable to aqueous fluids after laser irradiation, the implant having a shape of open tubing optionally having a narrowed site (a neck); ii) implanting said tubular implant in the anterior chamber, extraocularly near the limbus or intraocularly at the level of anterior chamber angle; iii) attaching the free ends of said tubular implant to each other by means of a lock to form a closed loop, the lock optionally having a T-shape, the loop essentially having a toroidal shape; iv) fixing the position of said loop inside the eye, but under the conjunctiva or scleral flap, by suturing it to the sclera, optionally using said narrowed site (neck), wherein the loop is near the limbus of an eye, its part being located within the anterior chamber; v) if the loop comprises said T-shaped lock, connecting it with an extraocularly situated plate via a linear connector, the plate being sutured to the sclera, located outside the sclera/below scleral patch of donor tissue; vi) measuring the intraocular pressure (TOP) of said eye; vii) if said TOP is higher than required, irradiating the implanted device by laser whereby opening a part of said plurality of laser-sensitive spots on the surface of the loop; and viii) examining whether the step vii resulted in lowering the value of said TOP; ix) repeating steps vii and viii till the TOP value reached a desired value.

### BRIEF DESCRIPTION OF THE DRAWINGS

The above and other characteristics and advantages of the invention will be more readily apparent through the following examples, and with reference to the appended drawings, wherein:

FIG. 1. is a schematic view showing an eye and three devices with an absorption plate and one device without a plate in accordance with embodiments of the invention, and further showing the position of said devices in the eye relatively toward the anterior chamber angle, limbus and the iris;

FIG. 2. is a schematic view of a toroidal device in accordance with one embodiment of the invention, showing a closed circular tubing comprising an outer impermeable membrane and laser-sensitive spots;

FIG. 3. is a schematic view of a toroidal device in accordance with one embodiment of the invention, showing a closed circular tubing similar to that one in FIG. 2 connected to a drainage linear tube leading the fluid to an extraocularly situated absorption plate; situated incomprising an outer impermeable membrane and laser-sensitive spots

FIG. 4. is a schematic view of a toroidal device in accordance with one embodiment of the invention, showing a bagel-shaped device having a part situated within the anterior chamber, and a part situated outside of the eye; the picture shows optional preplaced openings on the extraocular part which drain fluids from anterior chamber outside; and

FIG. 5. is a schematic view of a toroidal device in accordance with one embodiment of the invention, showing two types of closures; FIG. 5A shows a closed loop tubular device without a linear member leading the fluid to an absorption plate; FIG. 5B shows a closed loop tubular device with a Y-shaped closure for connecting to a linear member leading the fluid to an absorption plate.

### DETAILED DESCRIPTION OF THE INVENTION

An intraocular drainage device has now been designed that enables to adjust the amount of the exiting liquid according to observed intraocular pressure values, so that glaucoma problems may be more easily managed than with known devices. The device has a torus-shaped outer surface comprising an impermeable and laser insensitive membrane on which a plurality of laser-sensitive spots are placed, potentially forming a plurality of exits for the eye fluid after being eventually opened by applying YAG laser light—according to the need. The laser-sensitive spots are preformed during manufacturing of the device and exist before implanting the device to the eye. Preferably, one or more devices are placed in the angle area of the eye suffering from glaucoma, near/through the limbus. More spots, preformed on the torus surface, are laser-irradiated, resulting in higher permeability of the toroidal device or devices, providing higher fluid flow. This enables fine regulation of the drainage, as well as of the intraocular pressure. Known laser-insensitive and laser-sensitive materials can be employed in manufacturing devices according to the invention. Only for the sake of example, laser-sensitive polyesters may be mentioned here, but any other suitable material may be employed; in another aspect, the employed polymer may be processed to be thinned on certain areas, for example in “chessboard” manner. In one embodiment, the size of the spots, from which the liquid-releasing holes are formed, is lower than dimensions of typical contaminating microorganisms. The number of pre-formed spots increases with their decreasing surface in a simple relation enabling certain liquid volume to be drained, as understood by a skilled person.

In some embodiments, the device is implanted already with a part of the holes being preformed. In other embodiments, the device works without the drainage tube and/or without the absorbance plate, for example as trabeculectomy accessory. In still other embodiments, more than one device is implanted, whereby increasing the drainage efficiency or substituting a failed device. The invention may provide help in certain cases of closed-angle glaucoma.

The inner volume of the device, whether formed of a porous material or whether hollow, transports the liquid from a site of higher water thermodynamic activity to a site of lower water thermodynamic activity. The driving force may comprise the pressure difference, wicking phenomena, etc., but the final effects include lowering the damaging overpressure inside the eye. The device according to the invention drains fluid subconjunctivally, either under the conjunctiva or deeper.

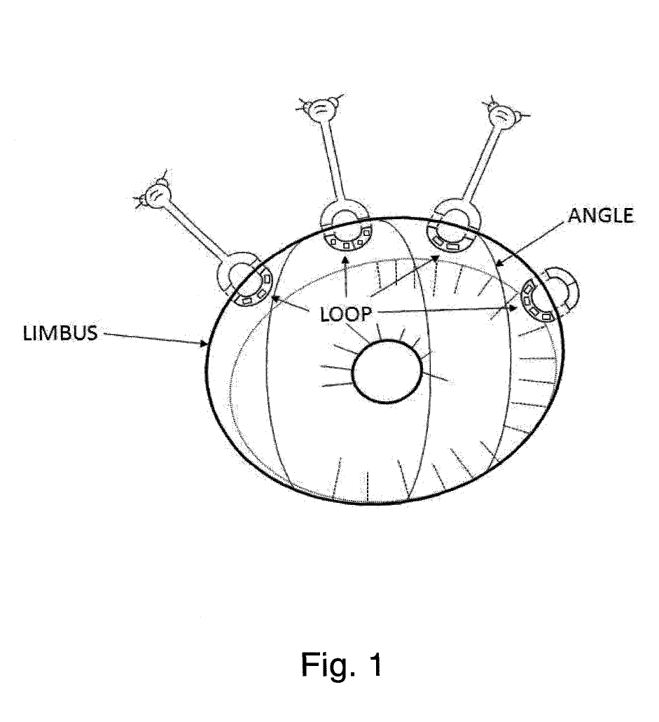

In a preferred embodiment of the invention, the device consists of a specially designed drainage loop with preplaced opening points to be later opened by laser, the shape of the device being essentially toroid. In other preferred embodiment of the invention, the device consists of two parts, the first comprising a toroid drainage loop with opening points as described above, and the second part being a drainage container or collector/drainage plate; usually, the plate is connected with the toroid structure by a linear connector tube. FIG. 1 schematically shows both cases.

The device will preferably be implanted in patients with uncontrolled glaucoma and/or in patient with failed previous treatments including surgery and laser. The main features of a preferred device according to the invention include a looped drainage tube, drainage tube comprising laser-compatible wall membranes with laser-sensitive spots (for example due to the material type or due to the thinner structure) to eventually provide openings. In one embodiment, the device may comprise a bagel-shaped drainage plate with or without scleral patching from a donor. The scleral patch may be preferred in cases when there is not enough conjunctiva tissue to cover the implant or when the implant is too large.

An important feature of the device of the invention is that it has a fixed position within the eye, it is placed immovably and stably, without being prone to known complications of tube devices. After placing the device within the eye, the surface area of created openings may be gradually increased to enhance the drained volume in accordance with the measured intraocular pressure, enabling to adjust the permeability of the device exactly as needed in a very flexible and sensitive way (clinical titration). The drainage capacity of the device can be affected by several parameters, including the number of laser-sensitive spots per square centimeter of the torus, the average diameter of the spot, the number of implanted devices, the type of eventual drainage container (such as absorption plate), the type of the eventual connector between the plate and the toroid, the site of location of the absorption plate on the eye body, the thickness of the toroid wall, the material of the toroid surface, the material and structure (porosity) of the toroid inner volume, the diameter of the tube making the toroid, the size of the toroid body, the fraction of irradiated laser-sensitive spots on the torus, etc.

A person skilled in handling glaucoma will appreciate that various parameters combinations may provide various problems solutions. In some applications, bagel-shaped device will be implanted to transfer the eye fluid out of the eye, and the device will be laser irradiated to open the preformed spots, in several stages to gradually increase the filtered volume up to the value exactly needed for a desired steady state; thus, a laser-assisted titration of the filtration rate is provided, which can help to manage the intraocular pressure in a more flexible and sensitive way.

The means for surgical treatment of glaucoma according to the invention are relatively less invasive than known means when performed without a plate, and less irritating than known means when performed without a plate or donor patches.

During the surgery, the practitioner usually makes two perforations in the limbus through which the device is passed and locked. Usually, scars form which can make pressure on the device. In the case of flexible fibers, the fibers can become easily compressed thus compromising filtration rate. The device according to the invention is preferably made of a relatively rigid material that can support the pressure exercised by the scar or other structures.

The instant device will preferably be used with refractory glaucomas and for eyes in which there is a high probability for trabeculectomy failure. Refractory glaucomas comprise, for example, uveitis, steroid-induced, neovascular, aphakic, ICE-syndrome, post-keratoplasty, associated with retinal or corneal surgery, associated with pseudophakia, aphakia, trauma, epithelial and fibrous downgrowth, aniridia, and iridocorneal endothelial syndrome. Known glaucoma implants comprise a drainage tube through which aqueous humor drains from the anterior chamber to an aqueous collecting plate that is sutured posteriorly. In the known devices, the tube has a single intraocular opening and the inside of the anterior chamber is fixed only at the level of the chamber angle: the end of the tube is not fixed and may touch near structures—cornea, iris and lens. Overfiltration and underfiltration are unpredictable negative outcomes. Free end of the tube is prone to dislocation and tube-related complication (i.e. corneal edema, cataract, hemorrhage, extrusion, failure etc.). The bagel-shaped glaucoma drainage device of the invention, either standalone or combined with plate device with adjustable filtration rate, preclude the drawbacks of the known devices. Drainage device has a bagel shape design, comprising an impermeable and laser insensitive membrane with preplaced laser sensitive spots to be opened according to the required liquid flow by laser. In one embodiment of the invention, the device has two independent drainage systems that are separated by an impermeable wall. In case a first drainage system fails, for example is obstructed, a practitioner can open a second drainage system, thus allowing the passage of the liquid humour through it, thus avoiding an additional surgery or other procedures. Different device diameters, and different membrane types may be applied; the drainage plate may be circular (bagel-shaped), and may have a collector plate (namely plate).

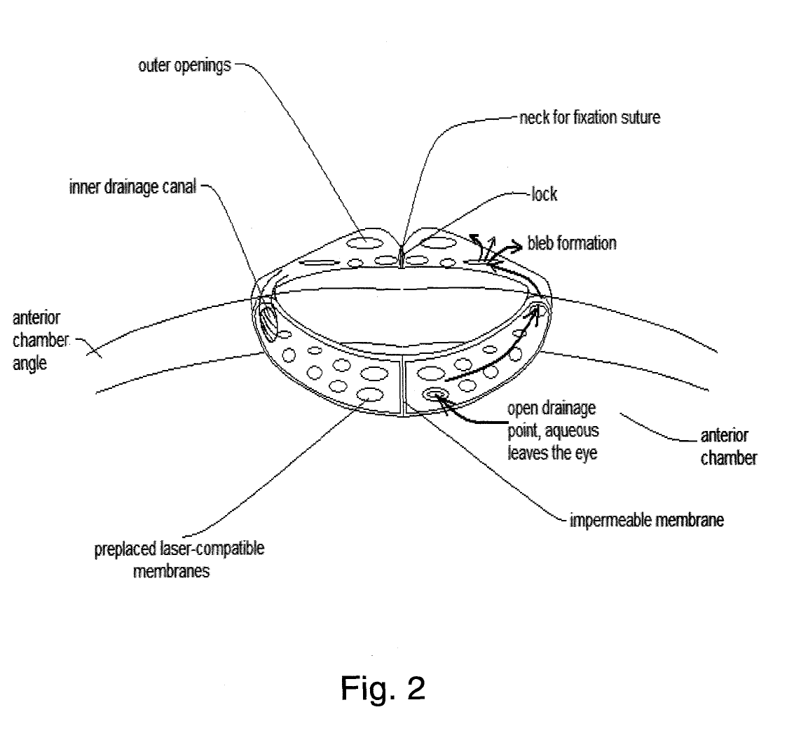

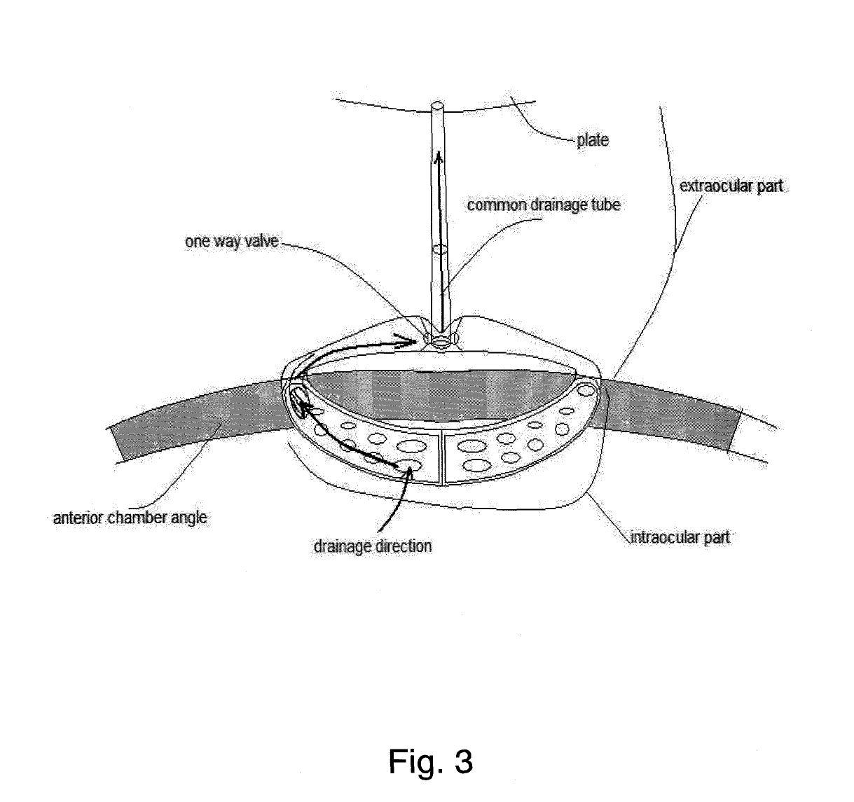

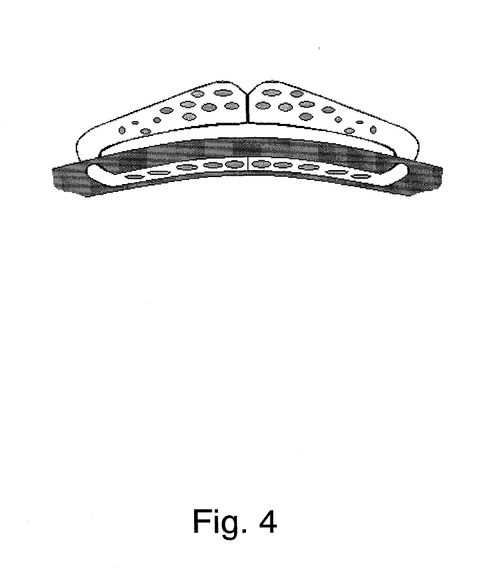

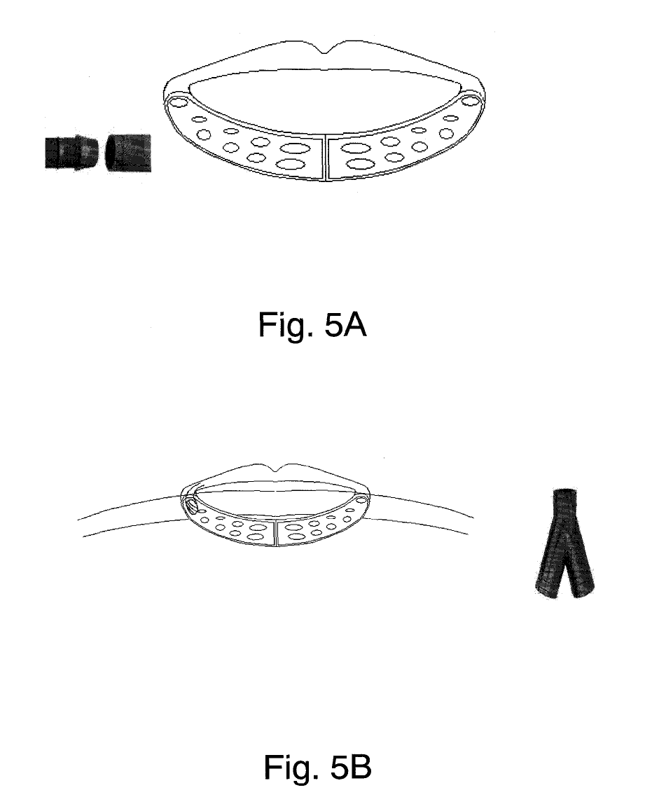

Parts of the device, for example extraocular part, may be coated with antifibroblasting material which prevents fibrovascular tissue formation around the device, such as, for example, mitomycin C and 5-FU. In one embodiment of the invention, the device is a silicone tubing which is implanted through two openings at the level of the anterior chamber angle of the eye. The implantation is guided by curved forceps, which helps to device to be inserted through one opening and to leave the eye through another. Subsequently, the ends of the tubing should be attached one to another and closed by lock, thereby rendering the device essentially toroidal shape (for example, see FIG. 5A); after the implantation the device is permanently sutured to sclera at the special neck of the device. The device efficiently drains the excessive fluid by itself (FIG. 2) or in connection with an absorption plate (FIG. 3). In the latter case, the lock may comprise a T-shaped or Y-shaped tube (FIG. 5B). In one embodiment, the base curve of the scleral part of the device (i.e. extraocular part) may have shape identical to the curve of the outer corneal curvature (i.e. limbal curvature) to be parallel to the anterior angle, and to allow exact fit of the device. In some embodiments, the intraocular part of the device can have the curvature reversed to the limbus curvature (i.e. true bagel form) or curvature parallel to the extraocular part (parallel to the limbus), allowing the device to be located at the level of the chamber angle only, and not to enter the anterior chamber deeply (FIG. 4). Outer parts may efficiently function as one-way valves to protect the eye from back flow of the fluids (FIG. 4). Extraocular and intraocular parts may have similar curves (parallel), so that there is no protrusion of the device to anterior chamber. The implantation may be located as subconjunctival, or below a scleral flap, with or without a donor patch. The device can be implanted in the lower part of the eye.

The device of the invention has no free movable ends and reduces usual tube-related complications considerably. The surgeon can adjust the filtration volume by means of the incremental increase of the openings effective surface—“titration”—while employing laser, for example YAG. Said clinical titration of drainage volume, together with non-irritating character of the torroidal shape, imparts to the device great flexibility and sensitivity, enabling its superior performance in reaching the goal of shunting aqueous fluid out of the anterior chamber, bypassing the trabecular meshwork to increase outflow, and lowering the IOP.

While this invention has been described in terms of some specific examples, many modifications and variations are possible. It is therefore understood that within the scope of the appended claims, the invention may be realized otherwise than as specifically described.

1. An intraocular drainage implant with adjustable drainage capacity, comprising an outer surface impermeable to aqueous fluids with a plurality of laser-sensitive spots permeable to aqueous fluids after laser irradiation.

2. The implant of claim 1, being a glaucoma drainage device.

3. The implant of claim 1, comprising a tubing whose ends are attached to each other by means of a lock to form a closed loop.

4. The implant of claim 1, essentially having a toroidal shape.

5. The implant of claim 1, for implanting in the anterior chamber near the limbus of an eye or at the level of the limbus.

6. The implant of claim 1, comprising a tubing closed in a loop and a neck for fixation suture.

7. The implant of claim 6, having a fixed position near the limbus of an eye or at the level of the limbus, wherein a part of said loop is located within the anterior chamber of said eye at the level of angle, and wherein said neck is sutured to the sclera of said eye.

8. The implant of claim 3, wherein said lock comprises a T-tube connected to a linear tube for shunting eye fluid from said loop to an extraocularly situated plate.

9. The implant of claim 6, comprising a tubing closed in a loop by a T-shaped lock, a linear tube connected to the loop via said lock, and an extraocularly situated plate connected with said linear tube, wherein a part of said loop is located within the anterior chamber of said eye, and wherein said neck and said plate are sutured to the sclera, the plate being either inside the sclera or onto the external surface of the sclera.

10. The implant of claim 6 for draining the eye fluid, wherein a part of said loop is located inside the anterior chamber near the limbus of said eye and a part of said loop is located outside said anterior chamber.

11. The implant of claim 6 with adjustable drainage capacity, comprising an outer surface impermeable to aqueous fluids with a plurality of laser-sensitive spots permeable to aqueous fluids after laser irradiation, wherein said eye fluid enters to said loop part which is located in the anterior chamber, via openings created from at least a part of said plurality of spots, and wherein said fluid is shunted via said loop out of the anterior chamber.

12. The implant of claim 11, being sequentially irradiated by laser in several steps, so that a greater part of said plurality of spots is opened to aqueous fluids after each step, thereby optimizing the volume of fluid to be continually removed from the eye.

13. A glaucoma drainage device for intraocular implanting, comprising a tubing having an outer surface impermeable to aqueous fluids with a plurality of laser-sensitive spots permeable to aqueous fluids after laser irradiation, wherein free ends of the tubing are attached to each other by means of a lock to form a closed loop, the device being implanted in the anterior chamber near the limbus of an eye and being sutured to the sclera to ensure its fixed position, said lock optionally comprising a T-tube to connect said loop with an extraocularly situated plate via an essentially linear connector, wherein a part of said loop is located within the anterior chamber of said eye shunting fluid out of the anterior chamber, the volume of said fluid being affected by the fraction of said spots being converted to openings by said irradiation, the fraction being optimized by sequentially irradiating the device in several increments.

14. A glaucoma drainage device according to claim 13 for use in managing refractory glaucoma.

15. A method of treating refractory glaucoma in an eye in need of such treatment, comprising
i) providing an intraocular tubular implant having an outer surface impermeable to aqueous fluids, with a plurality of laser-sensitive spots permeable to aqueous fluids after laser irradiation, the implant having a shape of open tubing optionally having a narrowed site (a neck);
ii) implanting said tubular implant in the anterior chamber near the limbus of an eye;
iii) attaching the free ends of said tubular implant to each other by means of a lock to form a closed loop, the lock optionally having a T-shape, the loop essentially having a toroidal shape;
iv) fixing the position of said loop outside the eye, but under the conjunctiva or scleral flap, by suturing it to the sclera, optionally using said narrowed site (neck), wherein the loop is near the limbus of an eye, its part being located within the anterior chamber;
v) if the loop comprises said T-shaped lock, connecting the lock with an extraocularly situated plate via a linear connector, the plate being sutured to the sclera, located either within or outside the sclera;
vi) measuring the intraocular pressure (TOP) of said eye;
vii) if said TOP is higher than required, irradiating the implanted device by laser whereby opening a part of said plurality of laser-sensitive spots on the surface of the loop;
viii) examining whether the step vii resulted in lowering the value of said TOP; and
ix) repeating steps vii and viii till the TOP value reached a desired value.
